(19) United States
(12) Patent Application Publication
(10) Pub. No.: US 2008/0234182 A1
(43) Pub. Date: Sep. 25, 2008

(54) BLOCKERS OF PORE-FORMING VIRULENCE FACTORS AND THEIR USE AS ANTI-INFECTIVES

(75) Inventor: Vladimir Karginov, Ashburn, VA (US)

Correspondence Address: HOGAN & HARTSON L.L.P., 1999 AVENUE OF THE STARS, SUITE 1400, LOS ANGELES, CA, 90067, US

(73) Assignee: Innovative Biologics, Inc., Herndon, VA (US)

(21) Appl. No.: 12/053,437

(22) Filed: Mar. 21, 2008

Related U.S. Application Data
(60) Provisional application No. 60/896,445, filed on Mar. 22, 2007.

(51) Int. Cl. A61K 38/12; A61K 31/724; A61K 31/357; C40B 40/00; C40B 30/00; A61P 31/04; A61P 31/12; C40B 30/02; C40B 50/14; A62D 3/30; A61K 31/409

(52) U.S. Cl. 514/9; 514/58; 514/410; 514/450; 588/299; 506/13; 506/30; 506/7; 506/8

(57) ABSTRACT

This invention provides a new generalized method for screening, identifying, selecting and designing symmetry-based compounds useful for blocking pores or prepores formed by pathogenic agents including bacteria, viruses, fungi, parasites, and other proteins capable of forming pores on cellular membranes as a step in the pathogenic mechanism of the agent. Also provided are pharmaceutical compositions, filtering devices, and treatment methods useful for preventing, delaying, or otherwise altering the pathogenesis of the pore-forming pathogenic agents.

### CROSS-REFERENCE TO RELATED APPLICATIONS

This application claims an invention which was disclosed in Provisional Application No. 60/896,445 filed Mar. 22, 2007, entitled “BLOCKERS OF PORE-FORMING VIRULENCE FACTORS AND THEIR USE AS ANTI-INFECTIVES”. The benefit under 35 USC §119(e) of the United States provisional application is hereby claimed. The above priority application is hereby incorporated herein by reference.

### FIELD OF THE INVENTION

The present invention relates to the development of symmetry-based small molecule blockers of pore-forming virulence factors and their use as anti-infectives.

### BACKGROUND OF THE INVENTION

Despite the significant advances made in antibiotics since Alexander Fleming first discovered penicillin in 1928, disease conditions caused by infective microbes (bacteria, viruses, parasites and fungi) continues to be a major medical problem. For example, Hepatitis C virus (HCV) is a major cause of cirrhosis and hepatocellular carcinoma; it infects over 3% of the worlds population. Currently available treatments include interferon and ribavirin but these are effective in only 50% of HCV infected individuals overall.

In another example, Influenza virus infections cause 3-5 million cases of severe illness and 250,000-500,000 deaths annually. In particular, the avian flu is now considered as a potential biological weapons threat. New strains of the influenza virus that are resistant to currently available drugs emerge every year, yet no effective and general means of countering these biological threats currently exist.

Anthrax is yet another example that has received significant media attention of late. Anthrax is a deadly disease and its causative agent Bacillus anthracis is considered as one of the most dangerous biological weapons. The absence of an effective treatment for postexposure inhalational anthrax is mostly due to the fact that antibiotics alone are not always helpful at this stage because of the accumulation of toxins. Again, no effective treatment has yet been approved to supplement intervention with antibiotics.

In another more mundane example, Staphylococcus aureus is one of the most common causes of serious hospital- and community-acquired infections. It is especially dangerous because of the high frequency of antibiotic-resistant strains. The search for new alternative ways to treat staphylococcal infections is considered an extremely important task.

Last, but not least, epsilon toxin (ETX) of Clostridium perfringens is one of the most lethal bacterial toxins. It is considered as a potential biological weapon and is included in the list of category B priority agents. No specific therapy exists for ε-toxin; therefore, a great need exists for the development of effective therapeutics against this biodefense toxin.

In view of the above, there exists an urgent need for new therapeutics strategies for combating these microbial and bioterror threats.

### SUMMARY OF THE INVENTION

Accordingly, one object of the present invention is to provide a method for designing and identifying new therapeutic agents useful for treating, preventing, or delaying a disease condition caused by pathogenic agents, in particular, pore-forming pathogenic agents.

It is also one object of the present invention to provide a new class of therapeutic agents and compositions thereof for treating pathogenic conditions caused by pore-forming pathogenic agents.

A further object of the present invention is to provide methods, compositions, and devices that are useful for defending against biological weapons.

These and other objects of the present invention, which will become more apparent in conjunction with the following detailed description of the preferred embodiments, either along or in combinations thereof, have been satisfied by the observation that many pathogens causes formation of transmembrane pores as part of its pathogenesis and the discovery that molecules having a symmetry and size that approximates the opening of the pore or its prepore are particularly effective in blocking the pores, and subsequently altering the progression of pathogenesis. Based on the observation and discovery of the present invention, the inventor has conceived and reduced to practice agents and compositions that are effective in blocking the pathogenic pores or prepores, methods for screening and identifying new compounds useful for treating, preventing, or delaying the pathogenesis, and methods for treating a patient utilizing the composition and devices of the present invention.

In a first aspect, the present invention provides a composition useful for treating, preventing, or delaying a disease condition in a subject caused by a pore-forming pathogenic agent. Embodiments according to this aspect of the present invention generally include a pharmaceutically acceptable carrier and a compound having a symmetry and size capable of fitting to an opening of the pore or its prepore for binding such that upon binding, the pore or prepore is blocked by the compound.

In a second aspect, the present invention also provides a method for treating, preventing, or delaying a disease condition in a subject by interfering with the pathogenesis of a causal agent of the condition. The pathogenesis of the causal agent include a step of forming a pore on the subject's cellular membrane. Embodiments according to this aspect of the present invention generally include the step of administering an effective amount of a pharmaceutical composition as described above to the subject.

In a third aspect, the present invention also provides a method for neutralizing a biological weapon. Embodiments according to this aspect of the present invention generally include a steps of providing a filtration device having a plurality of molecules with high binding affinity to an active agent of the biological weapon, followed by a step of filtering a material suspected of being exposed to the biological weapon through the filtering device. The active agent of the biological weapon is a pore-forming toxin, and the molecules have a structural symmetry and size capable of fitting to the pore or its prepore.

In a fourth aspect, the present invention also provides a device useful for screening or filtering pore-forming pathogenic agents. Embodiments according to this aspect of the present invention generally include a housing and a support medium contained therein, and pores or prepores formed by the pore-forming pathogenic agents immobilized on the support medium.

In a fifth further aspect, the present invention also provides a chemical library suitable for screening against a pore-forming target, and a method for forming such a library. Embodiments according to this aspect of the present invention generally include a plurality of molecules having a common chemical scaffold with a symmetry and size capable of fitting to the opening of the pore or prepore formed by the pore-forming target.

In a sixth aspect the present invention also provides a method for screening and selecting a drug candidate for treating a pathogenic condition caused by a pore-forming pathogenic agent capable of forming pores on cellular membranes. Embodiments according to this aspect of the present invention generally include the steps of: establishing and validating an assay for the pore-forming pathogenic agent; subjecting a symmetry-based chemical library as described above to the assay for testing and selecting the drug candidate.

Other aspects and advantages of the invention will be apparent from the following description and the appended claims.

### BRIEF DESCRIPTION OF THE DRAWINGS

FIG. 1 shows a schematic illustration of β-cyclodextrin molecule in comparison with the anthrax PA channel.

FIG. 2 shows protection of RAW 264.7 cells from LeTx-induced cell death by compound 14b. RAW 264.7 cells were incubated with different concentrations of the β-CD derivative with or without LeTx. Each experimental condition was performed in triplicate. Cell viability was determined by MTS colorimetric assay. Error bars represent standard deviations.

FIG. 3 shows typical tracks of ion conductance for PA channels reconstituted into planar lipid membranes. The downward arrow indicates the addition of AmPr□C (compound 5b) to the cis side of the membrane. The membrane was formed from diphytanoyl phosphatidylcholine; the membrane bathing solution contained 0.1M KCl, 1 mM EDTA at pH 6.6. Time averaging was 10 ms. The dashed lines show zero current level.

FIG. 4a shows protection of Fischer F344 rats from LeTx-induced death by AmPrβCD, Three groups of rats (n=3 per group) were injected IV with 10 μg LeTx (10 μg PA+10 μg LF) alone, or mixed with AmPrβCD (0.25 mg or 1.25 mg). A fourth group of rats (n=3) was pre-treated with 1.25 mg AmPrβCD and injected IV with LeTx after 30 min. Survival was monitored for each group continuously over 8 h and periodically for survivors over a period of 7 days. 4b shows protection of mice infected with B. anthracis.

FIG. 5 shows the 3D structure of the S. aureus α-hemolysin.

FIG. 6 shows protection of rabbit erythrocytes from α-HL action by compound 5040. Rabbit erythrocytes cells were incubated with different concentrations of the β--CD derivatives with or without α-HL. Each experimental condition was performed in triplicate. Hemolysis was determined calorimetrically at 415 nm. Error bars represent standard deviations. Rabbit Anti-Staphylococcal α-Toxin antibody (RAST) was used as a control.

FIG. 7 shows track of ion conductance for a single α-hemolysin channel reconstituted into a planar lipid membrane. The membrane was formed from diphytanoyl phosphatidylcholine; the membrane bathing solution contained 3M KCl at pH 6.6. Compound PP5040 was added to the cis side of the membrane. The dashed lines show zero current level.

FIG. 8 shows M2 tetramer with the same-scale molecule of tetrasaccharide cyclodextrin in the channel.

FIG. 9 shows M2 tetramer with the same-scale molecule of porphine in the channel.

FIG. 10 shows structures of α-, β-, and γ-cyclodextrins.

FIG. 11 shows protection of MDCK cells from ε-toxin-induced cytotoxicity by compound 5105. MDCK cells were incubated with different concentrations of compound 5105 with or without ε-toxin. Each experimental condition was performed in duplicates. Error bars represent standard deviations

### DETAILED DESCRIPTION

As set forth in the summary above, the present invention is based on the observation that many pathogenic agents form transmembrane pores as part of their pathogenesis and the discovery that certain molecules having symmetries and sizes approximating those of the pores or prepores are surprisingly effectively in altering the progression of pathogenesis. Accordingly, the present invention provides compounds, compositions, methods and devices that are useful for the treatment, prevention, and delay of pathogenic conditions caused by pore-forming pathogenic agents.

In a first aspect, the present invention provides a pharmaceutical composition useful for treating, preventing, or delaying a diseased condition in a subject caused by a pore-forming pathogenic agent, comprising a compound having a symmetry and size capable of fitting to an opening of the pore or its prepore for binding such that upon binding, the pore or prepore is blocked; and a pharmacologically acceptable carrier.

In the context of the present invention, the term “subject” refers to an individual organism which may be a human, an animal, or a plant.

In the context of the present invention, the term “preventing” is intended to encompass prevention of the onset of pathogenesis or prophylactic measures to reduce the risk of pathogenesis.

In some preferred embodiment, the compounds maybe a per-6-substituted cyclodextrin, a derivative thereof, a phorphyrin, porphine, a cyclic peptide or peptidomimetic, crown ether, or other symmetric molecules commonly known in the art.

As used herein, the term “symmetry-based” means that the selection and design of the compound is primarily based on symmetry considerations. For example, the pore opening of the PA toxin has a 7-fold symmetry. A symmetry-based selection or design will begin with a molecule having 7-fold symmetry or a symmetry that either approximates or is compatible with 7-fold symmetry. It is envisioned that application of symmetry principles can be applied loosely using a person's own intuitive sense or computer aided visualization tools. In some embodiments, rigorous application of symmetry considerations employing group theory is also contemplated. Mathematical descriptions and algorithms for symmetry similarity comparisons commonly known in the art may be employed. In a preferred embodiment, the compound has a symmetry identical to the symmetry of the opening of the pore or prepore.

The size of the compound is an important parameter. If the size is too big or too small the compound may not fit the opening. When the size is within a characteristic range, the matching symmetry forces that enhance molecular recognition such as proximity effect and multi-dentate effect may come into play, which may serve to give the molecule a strong binding affinity to the pore opening. To achieve excellent molecular recognition, the size (the longest axis) is preferably within 10% of the opening, more preferably within 5%.

To further enhance the binding affinity, the compound may also carrier surface charge or be a polar molecule. The charge or polarity is preferably complimentary to the charge or polarity of the opening of the pore or prepore.

Because molecules are dynamic entities, in preferred embodiments of the present invention, the compound should have limited conformational flexibility around the binding conformation so that the probability of the molecule binding the opening is enhanced. More preferably, the molecule should have a rigid scaffold. Exemplary symmetric and rigid scaffold may be selected from a-cyclodextrins, β-cyclodextrins, γ-cyclodextrins, porphyrins, and members of other commonly known cyclic and symmetric molecules, but are not limited thereto.

In a second aspect, the present the present invention also provides a method for treating, preventing, or delaying a disease condition in a subject by interfering with the pathogenesis of a causal agent of the condition, wherein the pathogenesis include a step of forming a pore on the subject's cellular membrane. Embodiments according to this aspect of the present invention generally include the steps of: administering an effective amount of a pharmaceutical composition as described in the first aspect of the invention to the subject.

In the context of the present invention, the terms “pathogenic causal agent” and “pathogenic agent” are used interchangeably and refers to the agent that causes the pathogenesis manifested in the subject. “The term qualifying phrase “pore-forming” when used together with “pathogenic agent” refers to those agents that form pores as step in the pathogenesis. In many instances, bacteria secrete proteins as virulence factors that form pores on the cellular membranes of the host.

The term “effective amount” as used in the context of the present invention is intended to qualify the amount of the active agent which will achieve the goal of improvement in disease severity and the frequency of occurrence while avoiding adverse effect. Each active agent will have a characteristic concentration that is optimal for treatment, which can be readily determined by routine pharmacological assays.

In some preferred embodiments according to this aspect of the present invention, the causal agent (i.e. the pathogen) may include a bacteria, a virus, a fungi, a parasite, or any combinations thereof, but are not limited thereto. The collective causal agents (bacterial, virus, fungi, and parasite) are also referred to herein as microbial pathogens.

Other causal agents may further include any pathogen known in the art that utilize pore-forming proteins as virulence factors.

Exemplary microbial pathogens may include Hepatitis C virus, an influenza virus, poliovirus, Sindbis virus, human respiratory syncytial virus, Semliki forest virus, Ross river virus, Clostridium perfringens, Clostridium difficile, Escherichia coli, Staphylococcus aureus, Bacillus anthracis, Aeromonas hydrophilia, Helicobacter pylori, Vibrio cholerae, Pseudomonas aeruginosa, Clostridium septicum, HIV and Bacillus sphaericus, Streptococcus pneumoniae, Streptococcus pyogenes, Clostridium botulinum, and Mycobacterium tuberculosis, but are limited thereto.

In another preferred embodiment, the causal agent is not a natural pathogen, but a weaponized pathogen such as one based on B. anthracis, S. aureus, and C. perfringens. This list is by no means exhaustive. It is envisioned that the method is applicable to patients who are at risk of being exposed to a biological weapon or those who are suspected and confirmed of having been exposed to the pathogen.

In a third aspect, the present invention also provides a method for neutralizing a biological weapon. Embodiments according to this aspect of the present invention generally include the steps of: providing a filtration device having a plurality of molecules with high binding affinity to an active agent of the biological weapon; and filtering a material suspected of being exposed to the biological weapon through the filtration device. In preferred embodiments, the active agent of the biological is a pore-forming toxin, and the molecules have a structural symmetry and size that are capable of fitting to the opening of the toxin pore or its prepore.

Materials such as food, air and water supply are common media by which biological weapon are passed onto the victims. In these embodiments of the present invention, filter devices based on molecules that have matching symmetry and size to the toxin pore or prepore may be advantageously used to filter food and air supply so as to reduce or eliminate the bio threat.

In a fourth aspect, the present invention also provides a device useful for screening or filtering pore-forming pathogenic agents. Embodiments according to this aspect of the present invention generally include: a housing and a support medium contained in the housing; and pores or prepores formed by the pore-forming pathogenic agents immobilized on the support medium.

In a preferred embodiment, the device is an affinity column. The housing for the device may be made from any suitable material known in the art. Exemplary material may include stainless steel, acrylic, ceramic, or any other inert structural material. The support medium may also be suitably chosen from common support medium known in the art such as polymer-based, or glass beads, but are not limited thereto. In other embodiments, the device may be in the form of a microfluidics instrument.

In a fifth aspect, the present invention also provides a chemical library suitable for screening against a pore-forming target, and a method for forming such a library. Embodiments according to this aspect of the present invention generally include: a plurality of molecules having a common chemical scaffold with a symmetry and size capable of fitting to the opening of the pore or prepore formed by the pore-forming target.

Suitable chemical scaffold may include cyclodextrins, porphyrins, and other cyclic and symmetric molecules known in the art, but are not limited thereto, so long as the selected scaffold has a symmetry that is similar or identical to the symmetry of the pore/prepore opening.

By extension, one skilled in the art will readily recognize that a method for forming such a library is also implied in this aspect of the present invention. Therefore, in one embodiment according to this aspect of the present invention, a method for forming a chemical library useful for screening against pore-forming pathogenic agents is also provided. In this embodiment, the method steps generally include the steps of: obtaining structural information of the pore opening; selecting a molecular scaffold having a symmetry and size capable of fitting to the pore or prepore opening; and populating the library with derivatives of the scaffold.

Exemplary structural information of the pore may include pore opening diameter, symmetry, and charge, but are not limited thereto.

Once a scaffold is selected, derivatization of the scaffold may be carried using any known chemistry technique in the art, including, but not limited to combinatorial chemistry techniques.

In a sixth aspect the present invention also provides a method for screening and selecting a drug candidate for treating a pathogenic condition caused by a pore-forming pathogenic agent capable of forming pores on cellular membranes. Embodiments according to this aspect of the present invention generally include the steps of: establishing and validating an assay for the pore-forming pathogenic agent; subjecting a symmetry-based chemical library as described above to the assay for testing and selecting the drug candidate.

Exemplary pore-forming pathogens are as described in the second aspect above, but not limited thereto. The pore proteins may be isolated using methods and techniques commonly known in the art. The assay may be any biological or biochemical assaying technique commonly known in the art. For example, binding assays or enzymatic assays may all be advantageously used to determine an interaction between a test candidate compound and the target pore. Other emerging and future developed assay technologies such as microfluidics may also be advantageously used. The method is preferably performed iteratively to incrementally improve the candidate selection.

In a further embodiment of the method, computational design may also be brought to bear and to improve the efficiency and success rate of the selection process. Common computational methods known in the art include de novo design, structure based design, or virtual screening may all be advantageously used.

In de novo design, one may begin by using information of the pore opening as a starting point and design a potential inhibitor based on symmetry and size considerations. Several well-known tools for de novo design may be suitably used in this application. One exemplary de novo tool is SPROUT (see V. Gillet, A. P. Johnson, P. Mata, S. Sike, P. Williams, J. Comput.-Aided Mol. Design, 7 (1993) 127, the entire content of which is incorporated herein by reference). Once a promising compound is design, a real compound corresponding to the designed compound can then be selected from the symmetry library for assay.

In another embodiment, virtual screening may also be used to identify compounds that meet the selection criteria. Any commonly know virtual screening tools may be suitably used for this step. An exemplary virtual screening tool is AutoDock (see Morris, G. M., Goodsell, D. S., Halliday, R. S., Huey, R., Hart, W. E., Belew, R. K. and Olson, A. J. “Automated Docking Using a Lamarckian Genetic Algorithm and Empirical Binding Free Energy Function”, J. Computational Chemistry, (1998) 19: 1639-1662., the entire content of which is incorporated herein by reference). Exemplary criteria may include molecular symmetry, size, charge complimentarily, but are not limited thereto.

Once the candidate compounds have been selected, suitable biological assays may be performed to determine and validate the activity of the selected candidates.

In yet another embodiment, structure-based design may be used in conjunction with actual screening in an iterative process. For example, in a first iteration, a plurality of weak candidates may be selected. Their structural features may then be analyzed by computational methods. One exemplary method is 3D-QSAR. A number of tools for performing such analysis is commercially available. One exemplary tool for performing such analysis is CATALYSTS from Accelrys (Accelrys, Inc., San Diego, Calif.). Based on such an analysis, the weak candidates may be optimized and improved in their activity.

Having generally described the various aspect and exemplary embodiments of this invention, a further understanding can be obtained by reference to certain specific examples which are provided herein for purposes of illustration only and are not intended to be limiting unless otherwise specified.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the attached claims.

### EXAMPLES

The inventor had previously made the surprising discovery that per-substituted derivatives of β-cyclodextrin (β-CD) having a sevenfold symmetry could block the heptameric pore formed by Bacillus antracis protective antigen (PA) and were protective against anthrax lethal toxin action in cell-based assays and in animal tests. Preliminary toxicity and efficacy tests in mice challenged with Bacillus anthracis also demonstrated the protective properties of β-cyclodextrin derivatives. Drawing on this success, the inventor further conceived of the approach as a general method in the present invention. The extent to which this symmetry-inspired approach of new inhibitor design may be applicable is farther investigated.

### 1. Seven-Fold Symmetry Library Design and Screening for PA Pore Blocker

Anthrax toxin, which plays a key role in anthrax infection, is formed by three polypeptides: protective antigen (PA) which either combines with lethal factor (LF) to form lethal toxin (LeTx), or with edema factor (EF) to form edema toxin (EdTx). LF and EF are enzymes that target substrates within the cytosol; PA provides a heptameric transmembrane pore to facilitate LF and EF transport into the cell (ref 1-3).

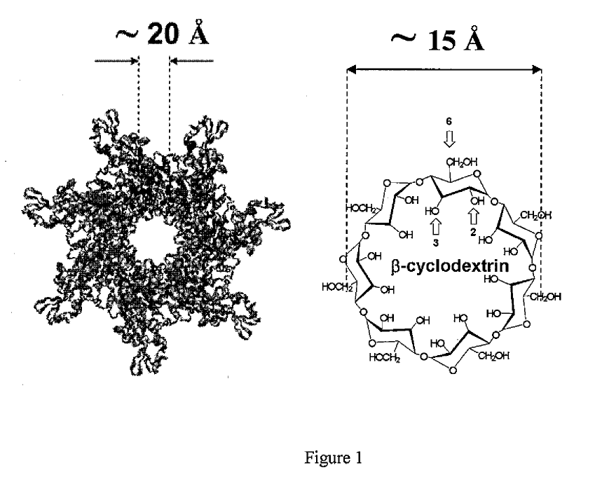

Guided by the seven-fold symmetry of the PA pore, cyclic molecules of seven-fold symmetry using β-cyclodextrin (β-CD) were designed, synthesized and tested as a starting molecule. β-CD molecules per-substituted with positively charged groups were also suggested as potentially effective blockers of the PA pore because the lumen of the PA pore is mostly negatively charged (ref 4) (FIG. 1). The hydroxyls at positions 2 and 3 form hydrogen bonds and are required to keep the molecule rigid, making 6-OH group a favorable site for modifications.

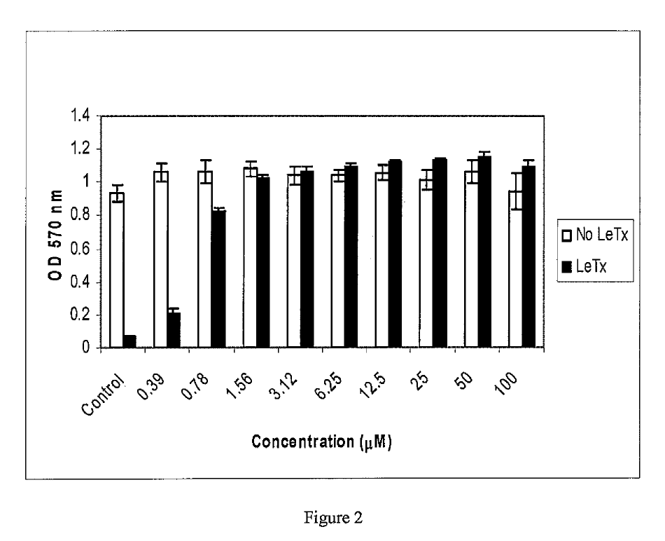

About 150 molecules representing a diverse group of β-cyclodextrin derivatives were designed and synthesized based on the above considerations. They were tested for the ability to inhibit cytotoxicity of LeTx. The extraordinary effectiveness of the approach was demonstrated by the fact that about 30% of the synthesized compounds displayed inhibiting activity at low- and sub-micromolar concentrations in cell-based assays (FIG. 2). These experiments also showed that in most cases the compounds were not toxic to RAW 264.7 cells up to a 100 μM concentration, while their IC₅₀ were as low as 0.6 μM.

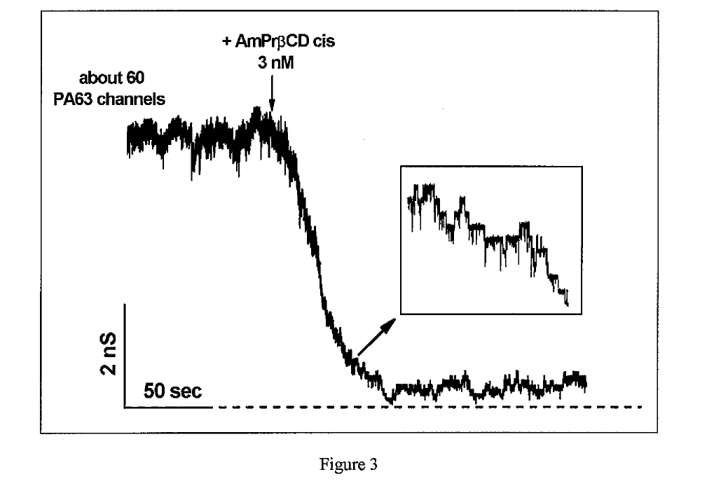

Several β-CD derivatives displaying inhibitory activity were tested for their ability to block conductance through the PA channels incorporated into the bilayer lipid membrane, and they blocked the PA pore at low nanomolar concentrations (FIG. 3). Activities of some of the tested compounds are presented in Table 1.

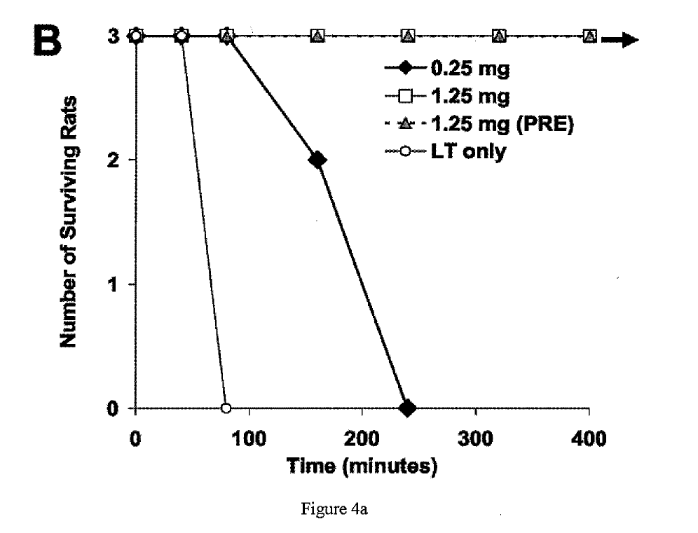

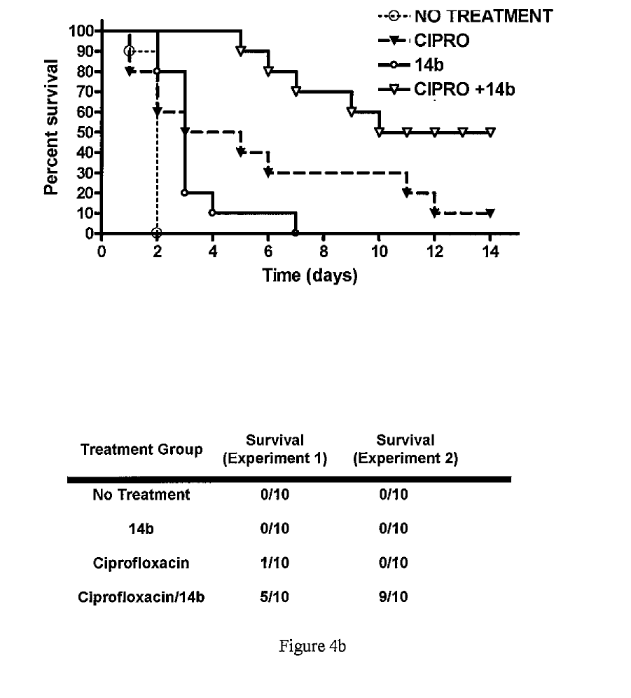

One of the most active compounds in vitro was tested in vivo and it completely protected rats from a deadly dose of anthrax lethal toxin (FIG. 4a). Fisher rats were injected with 10 μg LeTx alone, or mixed with 0.25 mg or 1.25 mg of AmPrβCD (compound 5b). Untreated rats died in about 80 min, rats treated with 1.25 mg of AmPrβCD were fully protected. The 0.25 mg dose did not protect the rats, but it extended their survival to approximately 200 minutes. Toxicity and efficacy tests in mice challenged with Bacillus anthracis also demonstrated the protective properties of β-cyclodextrin derivatives (FIG. 4b).

### 2. Broad Applicability of Symmetry-Based Inhibitor Screening and Design Method

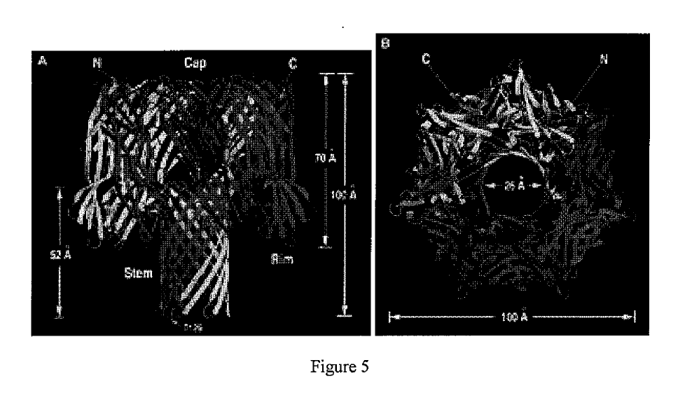

In the present invention, the inventor tested the broader applicability of the symmetry-based inhibitor design approach beyond anthrax and used α-toxin of Staphylococcus aureus (also called α-hemolysin) as a test target. This toxin is required for S. aureus virulence. Similar to the PA pore, the α-toxin also forms a heptameric pore. The inventor screened the library of β-cyclodextrin derivatives against α-toxin. The 3D structure of the α-toxin pore has been resolved,³¹ allowing computer-assisted rational drug design (FIG. 5).

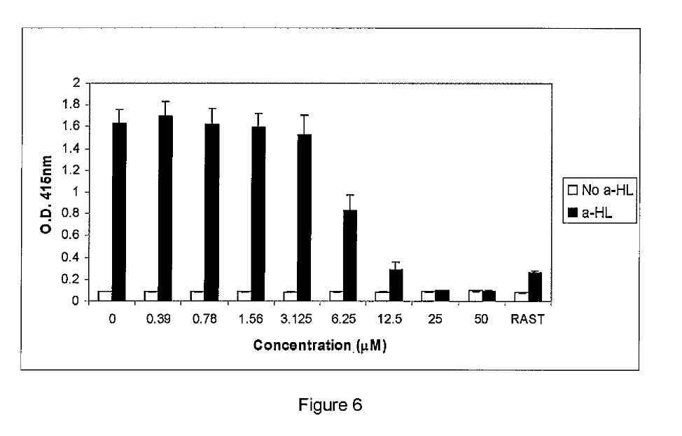

To screen the library for inhibitors of α-HL activity, a standard hemolysis assay was employed utilizing rabbit erythrocytes³² adapted to a 96-well plate format (FIG. 6).

Although the library was designed to block the pore formed by anthrax protective antigen, the screening revealed three compounds which inhibited hemolytic activity of α-HL at low micromolar concentrations (Table 2).

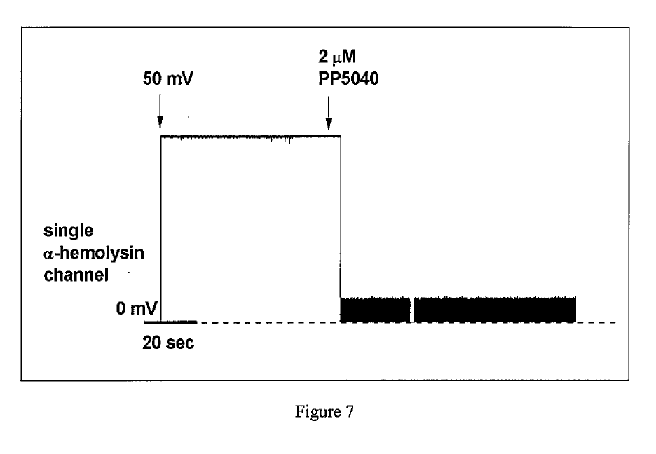

All of them are amino acid derivatives carrying protective groups (compounds 5040, 5046 and 5051), while compounds with fully deprotected amino acid groups did not display a potent inhibiting activity in this assay. Of these compounds only one derivative (compounds 5040) was active against both staphylococcal α-HL and anthrax LeTx in cell-based assays. Compound 5040 was tested for the ability to block ion conductance through the pores formed in artificial membranes by α-HL (FIG. 7) and it completely blocked the pore at 2 μM concentration.

This result suggests that the symmetry-based approach may be a fruitful strategy for designing new inhibitors against pore-forming toxins and pathogens.

For example, one of the important virulence factors in HCV pathogenesis is the protein p7, which forms heptameric trans-membrane channels in target cells similarly to anthrax PA and staphylococcal α-toxin. It was demonstrated by other investigators that the p7 ion channel can be blocked by amantadine, long alkyl chain imino-sugar derivatives, and amiloride compounds.

In view of the foregoing, a library of per-substituted β-CD derivatives is screened for p7 inhibitor activity. The experiment comprises i) establishing and validating assays for testing the ability of β-CD derivatives to block the pore formed by the p7 protein of HCV and to inhibit its cytotoxic activity; and ii) testing blocking and inhibitory activity of compounds from a representative library of per-substituted β-cyclodextrin derivatives and select the most potent compounds for further development as anti-toxin drugs.

The initial testing data and the structure information available for the p7 protein may be used in concert with computational chemistry to design additional β-CD derivatives with enhanced affinity to the p7 pore. The designed compounds will be synthesized and tested both in vitro and in vivo to develop new therapeutics against the hepatitis C virus.

This approach is of great utility in narrowing the search space for potential drug candidates and is expected to result in numerous products since it can be utilized for the discovery of new therapeutics against many other bacterial and viral pathogens that utilize pore-forming proteins as virulence factors. The list of the pathogens may include but is not limited to B. anthracis, S. aureus, H. pylori, C perfringens, V cholerae, C. septicum, hepatitis C virus, influenza virus and HIV.

For example, the inventor envisions utilizing the same approach for the development of new anti-influenza drugs. It has been shown that the well-known anti-influenza drugs amantadine and remantadine act by blocking the transmembrane channel formed by viral protein M2. They are recommended for use during influenza epidemics but the emergence of drug-resistant strains is a serious problem. Most of the known avian influenza virus strains are amantadine-resistant. The approach of the present invention should facilitate the design of new structurally distinct classes of M2 channel blockers that will be effective against amantadine-resistant strains of the influenza virus.

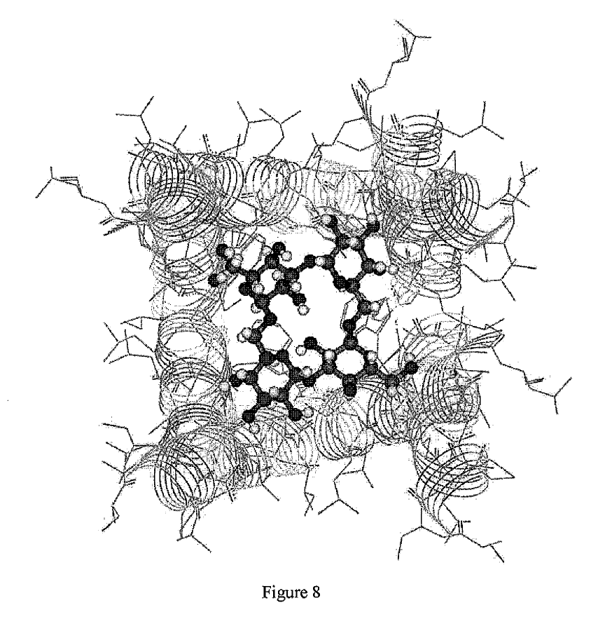

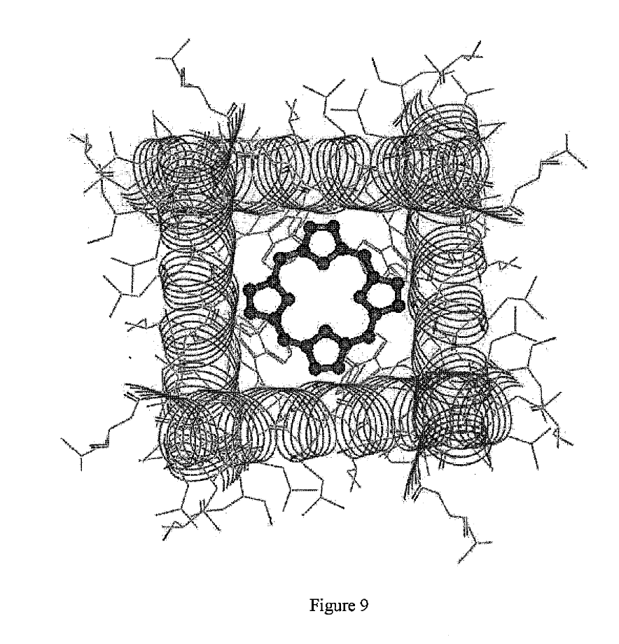

In an exemplary embodiment envisioned by the inventor, low molecular weight compounds with the potential to block the transmembrane channel formed by the influenza virus M2 protein will be designed and synthesized, and their anti-viral properties tested. Since the channel formed by the M2 protein is tetrameric, in accordance with methods of the present invention, the focus is on the design of molecules having four-fold symmetry such as derivatives of tetrasaccharide cyclodextrin or porphine. Preliminary computer modeling demonstrated that the outside diameters of these molecules are comparable with the diameter of the M2 channel (FIGS. 8 and 9). Therefore, these molecules will be selected as scaffolds for the development of high-affinity blockers of the M2 channel.

The experiment comprises i) establishing and validating assays for in vitro testing of the ability of the compounds to block the M2 channel and for in vivo testing of anti-viral activity; ii) design, using computer assisted docking, and synthesize a representative library of compounds in order to test their in vitro and in vivo activity; iii) utilizing initial testing data in concert with computational chemistry to design additional derivatives with an improved affinity for the M2 channel; and iv) preparing and testing a biased library of compounds. The most potent blockers are then selected as leads for a broader drug discovery program in order to find new drug candidates for the treatment of influenza infections.

### 3. Screening and Identification of Symmetry-Based ε-Toxin Pore Blockers

Using the symmetry-based approach of the present invention, compounds that inhibit C. perfringens ε-toxin action are identified for the inactivation of pore-forming toxins that is based on the blocking of the target pore with molecules having the same symmetry as the pore itself.

After effective inhibitors of ε-toxin are found in this feasibility study, the initial testing data and the crystal structure information available for C. perfringens ε-toxin will be used in concert with computational chemistry to design additional β-cyclodextrin derivatives with enhanced affinity to the ε-toxin pore. The designed compounds will be synthesized and tested in vitro and in vivo in order to find new therapeutics against C. perfringens ε-toxin.

Despite the effectiveness of the strategy, the chemical space is still large and effective use of the strategy will involve large-scale design, synthesis and screening of chemical libraries to select the best drug candidates for subsequent small animal studies.

To summarize, the inventor 1) has identified an important target—ε-toxin; 2) has developed an approach involving the blockage of bacterial toxin pores using molecules with the same symmetry as the pores; 3) has successfully tested the approach, which produced compounds with binding activity in the low nanomolar range, activity in the low micromolar range in cell-based assays, and protective activity in animal tests; and 4) has the starting molecule for the design (β-cyclodextrin) and its derivatives which have been well known in the pharmaceutical industry for decades.

The overall scheme of experiments consists of two steps. First, assays for testing the ability of compounds to block the s-toxin pore and to inhibit the cytotoxic effect of s-toxin are established and validated. Next, a representative library of β-cyclodextrin derivatives are screened to select the most potent blockers with activity in micromolar range, which are further tested for inhibitory activity using a cell-based assay.

### 4. Discussion

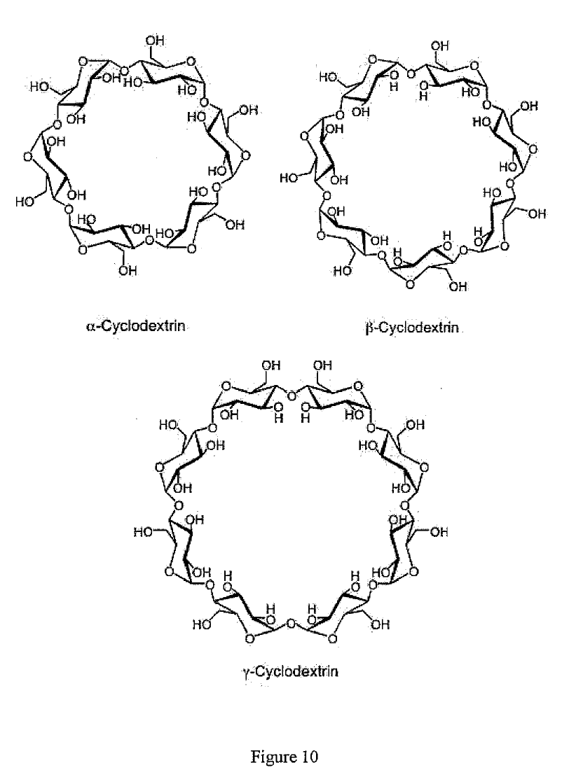

The inventor developed and validated an approach for the inactivation of bacterial pore-forming toxins which utilizes blocking of homooligomeric pores with molecules having the same symmetry as the pores. It was successfully tested on anthrax toxin (ref 5-6) and S. aureus α-hemolysin with the use of β-cyclodextrin derivatives as pore blockers. The α-, β- and γ-cyclodextrins are natural cyclodextrins, consisting of six, seven, and eight D-glucopyranose residues, respectively, linked by α-1,4 glycosidic bonds into a macrocycle (FIG. 10).

Cyclodextrins are known to encapsulate organic molecules in aqueous solution and have been widely used in pharmaceutical industries for decades to enhance solubility, bioavailability and stability of drug molecules. (ref 7-8) Although β-CD itself has low bioavailability (0.1-4% in rats), some of its derivatives have shown much better properties. For example, several β-CD derivatives demonstrated absorption levels up to 26% when they were administered in the rectum of rats; also adsorption of cyclodextrins from the gastrointestinal tract was detected. (ref 7) Most of the known cyclodextrins and their derivatives exhibit low toxicity and resistance to degradation by human enzymes and have GRAS (generally regarded as safe) status from the FDA. Methods for selective modifications of cyclodextrins are very well developed and offer excellent opportunities for the synthesis of various derivatives. (ref 9) The outside diameter of β-CD—15.3 Å—is comparable with the estimated diameter of the ε-toxin pore of about 20 Å.15 Thus, it is a surprising discovery of the present invention that cyclodextrins, in addition to their more mundane applications, has the potential of providing a new class of pharmaceutics.

### Experimental

### Screening Assays

Activation of Prototoxin. ε-prototoxin was obtained from Dr. Bruce McClane's lab at the University of Pittsburgh School of Medicine. The purified prototoxin was activated by incubation at 37° C. for 30 min with 0.1% trypsin in 0.02 M phosphate buffer (pH 8.0).

Cell-based assay. The assay is the MTS bioreduction cell viability assay, which can be potentially adapted for high throughput screening. A number of cell lines have been examined for sensitivity to ETX, and Madin Darby canine kidney (MDCK) cells have been identified to date as displaying the highest sensitivity to the toxin (ref 12-15). The assay protocol is presented below.

MDCK cells were cultured in Eagle's minimum essential medium containing Earle's salts, penicillin (100 units/ml), and streptomycin (100 μg/ml), supplemented with 10% heat-inactivated fetal bovine serum, in a cell culture incubator under 5% CO₂ at 37° C. Freshly trypsinized cells were cultured in 96-well microculture plates for 48 h to give monolayers. The medium was exchanged for 200 μl of minimum essential medium with or without a β-CD derivative, followed by the addition of 50 μl PBS containing ε-toxin. After 6 hour incubation, cell viability was determined using the MTS cell viability kit (Promega, Madison, Wis.). The absorbance, which is proportional to the number of viable cells, was read at 570 nm on a multi-well scanning spectrophotometer.

As a positive control in the above assays, the inventor used antibodies against ε-toxin that have been shown to be capable of protecting MDCK cells from ETX action. Petit et al. demonstrated that polyclonal anti-ETX antibodies or a monoclonal anti-epsilon antibody were capable of preventing toxin heptamerization in MDCK membranes (ref 16).

### Screening.

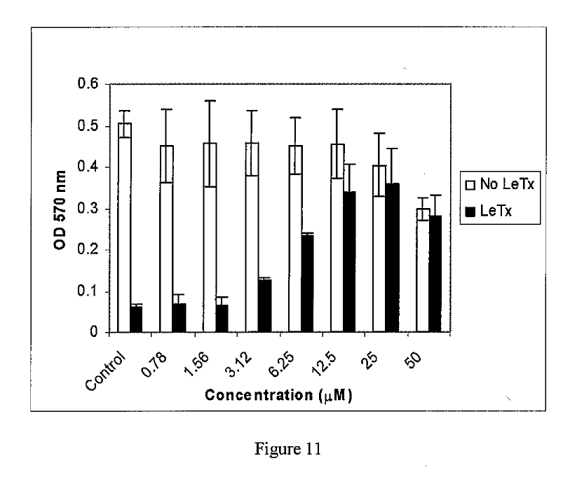

We have screened over one hundred β-cyclodextrin derivatives at a concentration of 50 μM using the cell-based assay. Compounds that showed at least 50% inhibition of ε-toxin cytotoxicity at a 50 μM concentration were selected. They were serially diluted and tested to determine the IC₅₀ values. Three structurally related compounds displayed dose-dependent inhibition of s-toxin cytotoxicity (FIG. 11, Table 3 Compounds 8-10).

The inventor has a library of 150 β-CD derivatives per-substituted at position 6 with various neutral, positively or negatively charged groups including amino, S-aminoalkyl, O-aminoalkyl, N-aminoalkyl, S-alkylguanidyl, O-alkylguanidyl, N-alkylguanidyl, n-alkyl, arylalkyl, aryl, heterocyclic rings, OSO₃Na and others. Also, β-CD derivatives that could be utilized in this project are available from companies and laboratories commercially producing cyclodextrins, such as Cyclodextrin Technologies Development, Inc. (Florida), CycloLab (Hungary), Cytrea Ltd. (Ireland) and others. The diversity of the derivatives has allowed the inventor to find a high number of compounds with the inhibitory activity at low- and sub-micromolar concentrations against anthrax, C. perfringens and staphylococcal toxins having very different primary structures and mechanisms of actions. The only common feature of the two toxins is the formation of heptameric pores in the membranes of target cells.

TABLE 1
Inhibition of LeTx by β-CD derivatives.

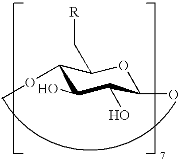

| | | | |
|---|---|---|---|
| | | Inhibition of cytotoxicity | Inhibition of trans-membrane conductance |
| # | R | IC₅₀ (μM) | IC₅₀ (nM) |

I. Hepta-6-aminoalkyl β-cyclodextrin derivatives

| | | | |
|---|---|---|---|
| 15 | NH₂ | 12.1 ± 3.5 | 32 ± 15 |
| 5a | S(CH₂)₂NH₂ | 7.8 ± 2.4 | 3.5 ± 0.9 |
| 5b | S(CH₂)₃NH₂ | 2.9 ± 1.0 | 0.6 ± 0.4 |
| 5c | S(CH₂)₄NH₂ | 5.1 ± 2.4 | 1.1 ± 0.5 |
| 5d | S(CH₂)₅NH₂ | 7.5 ± 2.4 | 3.8 ± 1.0 |
| 5e | S(CH₂)₆NH₂ | 0.6 ± 0.3 | 1.0 ± 0.4 |
| 5f | S(CH₂)₇NH₂ | 1.9 ± 1.1 | 4.6 ± 3.2 |
| 5g | S(CH₂)₈NH₂ | 0.3 ± 0.1 | 2.4 ± 1.0 |
| 5h | S(CH₂)₉NH₂ | 0.8 ± 0.1 | 14.7 ± 9.7 |
| 5i | S(CH₂)₁₀NH₂ | 2.6 ± 0.7 | 27.0 ± 17.0 |

II. Hepta-6-guanidinealkyl β-cyclodextrin derivatives

| | | | |
|---|---|---|---|
| 9a | 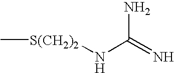 | 8.9 ± 6.0 | 5.3 ± 3.2 |
| 9b | 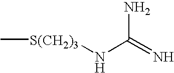 | 12.2 ± 2.9 | 12.6 ± 9.0 |
| 9c | 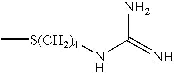 | 3.8 ± 2.3 | — |
| 9e | 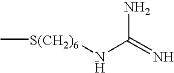 | 2.3 ± 0.4 | — |

III. Hepta-6-arylamine β-cyclodextrin derivatives

| | | | |
|---|---|---|---|
| 17 | 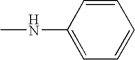 | >200 | — |
| 18 | 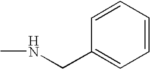 | 2.3 ± 1.2 | — |

IV. Hepta-6-alkylarylamine β-cyclodextrin derivatives

| | | | |
|---|---|---|---|
| 14a | 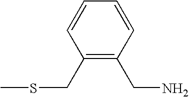 | 1.7 ± 0.4 | — |
| 14b | 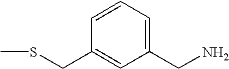 | 0.5 ± 0.2 | 0.07 ± 0.05 |
| 14c | 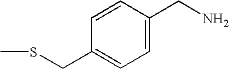 | 0.7 ± 0.5 | — |

TABLE 2
Inhibition of α-HL and LeTx by β-CD derivatives

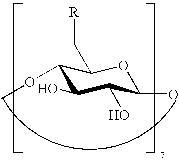

| | | | Inhibition | |
|---|---|---|---|---|
| | | | of LeTx | Inhibition |
| | | | cyto- | of α-HL |
| | | | toxicity | cyto- |
| | | | IC₅₀ (μM) | toxicity |
| | | | RAW | IC₅₀ (μM) |
| | Com- | | 264.7 | Red Blood |
| # | pound # | R | Cells | Cells |
| 1 | 5040 | 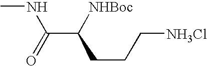 | 3.5 ± 2.2 | 5.6 ± 1.8 |
| 2 | 5041 | 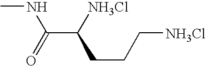 | >25 | >25 |
| 3 | 5046 | 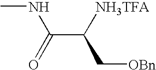 | >25 | 6.1 ± 2.4 |
| 4 | 5047 | 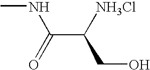 | >25 | >25 |
| 5 | 5051 | 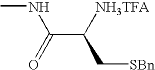 | >25 | 10.6 ± 3.0 |
| 6 | 5052 | 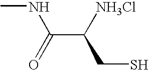 | >25 | >25 |

TABLE 3
Candidate symmetry-based pore blockers

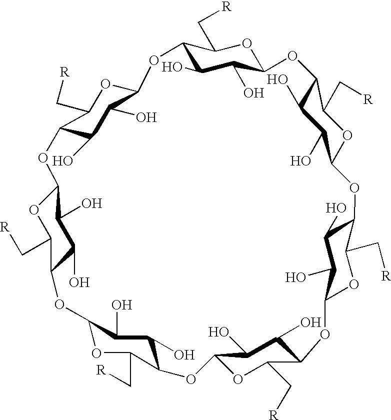

| | | |
|---|---|---|
| | | Inhibition |
| # | R | of cytotoxicity IC₅₀ (μM) |

I. β-cyclodextrin derivatives most active against anthrax toxin

| | | |
|---|---|---|
| 1 | 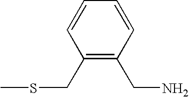 | 1.7 ± 0.4 |
| 2 | 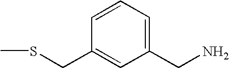 | 0.5 ± 0.2 |
| 3 | 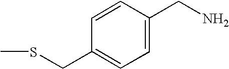 | 0.7 ± 0.5 |
| 4 | S(CH2)₈NH₂ | 0.3 ± 0.1 |

2. β-cyclodextrin derivatives most active against staphylococcal α-toxin

| | | |
|---|---|---|
| 5 | 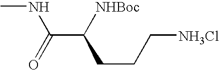 | 5.6 ± 1.8 |
| 6 | 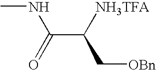 | 6.1 ± 2.4 |
| 7 | 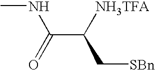 | 10.6 ± 3.0 |

3. β-cyclodextrin derivatives most active against C. perfringens ε-toxin

| | | |
|---|---|---|
| 8 | 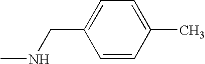 | 20 ± 10 |
| 9 | 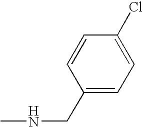 | 21 ± 9 |
| 10 | 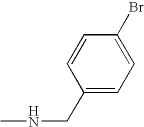 | 20 ± 1 |

### REFERENCES

The following references are each relied upon and incorporated by reference herein:
1. Brossier F, Mock M. Toxins of Bacillus anthracis. Toxicon 2001; 39: 1747-1755.
2. Collier R J, Young J A. Anthrax toxin. Annu Rev Cell Dev Biol 2003; 19: 45-70.
3. Moayeri M, Leppla S H. The roles of anthrax toxin in pathogenesis. Curr Opin Microbiol 2004; 7: 19-24.
4. Benson E L, Huynh P D, Finkelstein A, Collier R J. Identification of residues lining the anthrax protective antigen channel. Biochemistry 1998; 37: 3941-3948.
5. Karginov V A, Yohannes A, Robinson T M, Fahmi N E, Alibek K, Hecht S M. β-Cyclodextrin derivatives that inhibit anthrax lethal toxin. Bioorg Med Chem 2006; 14(1): 33-40.
6. Karginov V A, Nestorovich E M, Moayeri M, Leppla S I, Bezrukov S M. Blocking anthrax lethal toxin at the protective antigen channel by using structure-inspired drug design. Proc Natl Acad Sci USA 2005; 102(42): 15075-15080.
7. Uekarna K, Hirayama F, Tetsumi I. Cyclodextrin drug carrier systems. Chem Rev 1998; 98:2045-2076.
8. Davis M E, Brewster M E. Cyclodextrin-based pharmaceutics: past, present and future. Nat Rev 2004; 3:1023-1035.
9. Khan A R, Forgo P, Stine K J, D'Souza V T. Methods for selective modification of cyclodextrins. Chem Rev 1998; 98:1977-1996.
10. Nagahama M, Michiue K, Sakurai J. Membrane-damaging action of Clostridium perfringens alpha-toxin on phospholipid liposomes. Biochim Biophys Acta 1996; 1280(1): 120-6.
11. Nagaharna M, Hara H, Fernandez-Miyakawa M, Itohayashi Y, Sakurai J. Oligomerization of Clostridium perfringens epsilon-toxin is dependent upon membrane fluidity in liposomes. Biochemistry 2006; 45(1): 296-302.
12. Hambrook J L, Lindsay C D, Hughes N. Morphological alterations in MDCK cells induced by exposure to Clostridium perfringens epsilon-toxin. Biochem Soc Trans 1995; 23(1): 44S.
13. Lindsay C D. Assessment of aspects of the toxicity of Clostridium perfringens epsilon-toxin using the MDCK cell line. Hum Exp Toxicol 1996; 15: 904-908.
14. Payne D W, Williamson E D, Havard H, Modi N, Brown J. Evaluation of a new cytotoxicity assay for Clostridium perfringens type D epsilon toxin. FEMS Microbiol Lett 1994; 116: 161-167.
15. Shortt S J, Titball R W, Lindsay C D. An assessment of the in vitro toxicology of Clostridium perfringens type D epsilon-toxin in human and animal cells. Hum Exp Toxicol 2000; 19: 108-116
16. Petit L, Gibert M, Gillet D, Laurent-Winter C, Boquet P, Popoff M R. Clostridium perfringens epsilon-toxin acts on MDCK cells by forming a large membrane complex. J Bacteriol 1997; 179: 6480-6487.

What is claimed is:

1. A pharmaceutical composition useful for treating, preventing, or delaying a disease condition in a subject caused by a pore-forming pathogenic agent, comprising:
a compound having a symmetry and size capable of fitting to an opening of the pore or its prepore for binding such that upon binding, the pore or prepore is blocked; and
a pharmacologically acceptable carrier.

2. The pharmaceutical composition of claim 1, wherein said compound is a per-6-substituted cyclodextrin, a derivative thereof, a phorphyrin, porphine, a cyclic peptide or peptidomimetic, or crown ether.

3. The pharmaceutical composition of claim 1, wherein said compound has a symmetry identical to the symmetry of the opening of the pore or prepore.

4. The pharmaceutical composition of claim 1, wherein said compound has a size within 10% of the pore diameter.

5. The pharmaceutical composition of claim 1, wherein said compound has a size within 5% of the pore diameter.

6. The pharmaceutical composition of claim 1, wherein said compound has a charge distribution complimentary to the charge of the opening of the pore or prepore.

7. The pharmaceutical composition, wherein the compound has a rigid scaffold.

8. The pharmaceutical composition, wherein said pathogenic agent is B. anthracis and said compound is selected from compounds 1-4 shown in Table 3, or any compound shown in Table 1 except for compound 17.

9. The pharmaceutical composition, wherein said S. aureus and said compound is selected from compounds 5-7 of Table 3.

10. The pharmaceutical composition, wherein said C. perfringens and said compound is selected from compounds 8-10 of Table 3.

11. A method for treating, preventing or delaying a disease condition in a subject by interfering with the pathogenesis of a causal agent of the condition, comprising:
administering an effective amount of a pharmaceutical composition according to claim 1 to the subject,
wherein said pathogenesis comprises a step of forming a pore on the subject's cellular membrane.

12. The method of claim 11, wherein said causal agent is one selected from a bacteria, a virus, a fungi, a parasite, or combinations thereof.

13. The method of claim 11, wherein said causal agent is a pathogen utilizing pore-forming proteins as virulence factors.

14. The method of claim 11, where said casual agent is one selected from Hepatitis C virus, an influenza virus, poliovirus, Sindbis virus, human respiratory syncytial virus, Semliki forest virus, Ross river virus, Clostridium perfringens, Clostridium difficile, Escherichia coli, Staphylococcus aureus, Bacillus anthracis, Aeromonas hydrophilia, Helicobacter pylori, Vibrio cholerae, Pseudomonas aeruginosa, Clostridium septicum, HIV and Bacillus sphaericus, Streptococcus pneumoniae, Streptococcus pyogenes, Clostridium botulinum, and Mycobacterium tuberculosis.

15. The method of claim 11, wherein said casual agent is B. anthracis and said pharmaceutical composition is according to claim 8.

16. The method of claim 11, wherein said causal agent is S. aureus and said pharmaceutical composition is according to claim 9.

17. The method of claim 11, wherein said causal agent is C. perfringens and said pharmaceutical composition is according to claim 10.

18. The method of claim 11, wherein said causal agent is a biological weapon and said administering step is performed on a subject who is at risk of being exposed to the biological weapon or is suspected of having been exposed.

19. The method of claim 18, wherein:
A: said biological weapon is one based on B. anthracis, and the said pharmaceutical composition is one containing a compound selected from compound 1-4 of Table 3; or
B: said biological weapon is one based on S. aureus, and said pharmaceutical composition is one containing a compound selected from compound 5-7 of Table 3; or
C: said biological weapon is one based on C. perfringens, and said pharmaceutical composition is one containing a compound selected from compounds 8-10 of Table 3.

20. A device useful for screening or filtering pore-forming pathogenic agents, comprising:
a housing and a support medium contained therein; and
pores or prepores formed by the pore-forming pathogenic agents immobilized on the support medium.

21. A method for neutralizing a biological weapon, comprising:
providing a filtration device according to claim 20, wherein said pore-forming pathogenic agent is the biological weapon; and
filtering a material suspected of being exposed to the biological weapon through the filtration device,
wherein said active agent of the biological weapon is a pore-forming toxin, and said molecules having a structural symmetry and size capable of fitting to the pore or its prepore.

22. A symmetry-based chemical library suitable for screening against a pore-forming target, comprising:
a plurality of molecules having a common chemical scaffold with a symmetry and size capable of fitting to the opening of the pore or prepore formed by the pore-forming target.

23. A method for forming a symmetry-based chemical library as set forth in claim 22, comprising:
obtaining structural information of the pore opening, wherein said structural information include diameter of the opening and symmetry of the opening;
selecting a molecular scaffold having a symmetry and size capable of fitting to the pore opening; and
populating the library with derivatives of the scaffold.

24. A method for screening and selecting a drug candidate for treating a pathological condition caused by a pore-forming pathogenic agent capable of forming pores on cellular membranes, comprising:
establishing and validating an assay for the pore-forming pathogenic agent;
subjecting a library of potential candidate compounds to the assay for testing and selecting the drug candidate, wherein said library is a symmetry-based chemical library according to claim 22.

25. The method of claim 24, further comprising a step of computational design, wherein said computational design is one selected from:
A: de novo design using as a starting point the same molecular scaffold as the symmetry-based chemical library and then testing the designed molecule in the assay;
B: structure-based design using a candidate compound identified from the testing step as the starting point; or
C: virtual screening wherein a virtual chemical library corresponding to the symmetry-based chemical library is used to identify and select candidate compounds from the library for testing.
